(19) United States
(12) Patent Application Publication
(10) Pub. No.: US 2019/0285615 A1
(43) Pub. Date: Sep. 19, 2019

(54) METHODS AND ARTIFICIAL TUMORS FOR VALIDATING AN ANTITUMOR IMMUNE RESPONSE

(71) Applicants: NantBio, Inc., Culver City, CA (US); Nant Holdings IP, LLC, Culver City, CA (US)

(72) Inventors: Shahrooz RABIZADEH, Culver City, CA (US); Kayvan NIAZI, Encino, CA (US); Nicholas J. WITCHEY, Culver City, CA (US)

(73) Assignees: NantBio, Inc., Culver City, CA (US); Nant Holdings IP, LLC, Culver City, CA (US)

(21) Appl. No.: 16/464,824

(22) PCT Filed: Dec. 14, 2017
(86) PCT No.: PCT/US2017/066269

§ 371 Date: May 29, 2019

Related U.S. Application Data
(60) Provisional application No. 62/436,180, filed on Dec. 19, 2016.

(51) Int. Cl. G01N 33/50; C12N 5/09

(52) U.S. Cl. CPC G01N 33/5011; C12N 5/0693

(57) ABSTRACT

The invention provides methods for validating an antitumor response by growing and maintaining artificial 3D tumors under conditions that replicate the microenvironment of the naturally occurring parent tumor as found, in situ, in a subject diagnosed with a tumor or cancer. The artificial 3D tumors are obtained from the subject and once established are tested for susceptibility to proposed anticancer treatments, such as treatments personalized to the subject.

### FIELD OF THE INVENTION

The field of the invention relates to methods for validating an antitumor response by growing and maintaining artificial 3D tumors under conditions that replicate, as much as possible, the microenvironment of the naturally occurring parent tumor as found, in situ in a subject diagnosed with a tumor or cancer.

### BACKGROUND OF THE INVENTION

The background description includes information that may be useful in understanding the present invention. It is not an admission that any of the information provided herein is prior art or relevant to the presently-claimed invention, or that any publication specifically or implicitly referenced is prior art.

All publications, patents, and patent applications recited herein are incorporated by reference to the same extent as if each individual publication, patent, or patent application were specifically and individually incorporated by reference. Where a definition or use of a term in an incorporated publication, patent, or patent application is inconsistent or contrary to the definition of that term provided herein, the definition of that term provided herein applies and the definition of that term in the reference does not apply.

Despite recent advances in the diagnosis and treatment of cancer, many types of cancers remain difficult to drive into long term remissions. Currently, one promising avenue of research is the individualized therapy of cancers. The concept is based on the observation that each cancer, in each individual patient or subject, is in some ways unique, and if the therapy is tailored to address or target the specific damaged genetic and/or regulatory cellular pathway in a specific tumor, the prospects of a successful treatment will be enhanced. Thus, anticancer vaccines, or other types of immune therapies for eliciting an immune response to a patient specific tumor, or anticancer pharmaceutical regimens tailored to a specific tumor found in a specific individual subject, are under investigation and have shown some early dramatic successes in improving clinical outcomes for subjects diagnosed with cancer.

However, individualized, subject specific anticancer treatments are very costly in time, money, laboratory resources, and in the efforts of the individual subjects receiving the treatment. Thus, there remains a longstanding need in the art for cost effective, efficient and relatively rapid methods of verifying or validating the effectiveness of a proposed individualized anticancer therapy, including immunotherapy, in eliciting an antitumor immune response in a subject. By way example, the anticancer therapeutic is a vaccine, biologic and/or pharmaceutical anticancer agent, radiation therapy, and/or a combination thereof.

### SUMMARY OF THE INVENTION

Thus, the invention is directed to a method of validating a treatment within an artificial tumor microenvironment comprising:
(a) obtaining a viable tumor sample from a tumor present in a subject at an anatomical location;
b) acquiring tumor microenvironment parameters from the subject's tumor, i.e., the donor subject, from the viable tumor sample obtained from the donor subject, or from tissue surrounding the anatomical site of the tumor in the donor subject;
(c) adjusting an artificial culture medium according to the tumor microenvironment parameters;
(d) treating at least a portion of the tumor sample in the adjusted artificial culture medium with at least one treatment; and
(e) quantifying an effect of the at least one treatment

Quantifying step (e) can be measured with respect to at least one non-tumor cell within the portion of the tumor sample, or alternatively, can be measured with respect to the tumor or tumor cell parameters determined at the start of the treating step.

The viable tumor sample includes a plurality of suspended cells or includes a sample of tumor tissue or other sample configurations.

The step of obtaining the tumor sample includes, for example, obtaining samples of the vasculature of the tumor sample, at or adjacent to the in vivo anatomical location of the tumor.

The step of acquiring tumor microenvironment parameters for the subject's tumor or the acquired tumor sample includes, one or more of taking a metabolomics measurement of the viable tumor sample, and/or acquiring omics data of the viable tumor sample. The omics data is derived from at least one of whole gene sequencing (WGS), whole exome sequences (WES), transcriptome profiling including mRNAs, non-coding RNAs and small RNAs (RNAseq), and profiling the proteins expressed by the tumor (proteomics analysis). The omics data are preferably processed and analyzed using BAMBAM methods.

The step of acquiring tumor microenvironment parameters for the subject's tumor or the acquired tumor sample further includes, one or more of taking an in vivo or an in vitro measurement of the tumor microenvironment parameters such as , taking a vasculature measurement, taking an exosome measurement, measuring an oxygen parameter, i.e., concentration or partial pressure, a pH parameter, i.e., the antilog of the hydrogen ion concentration in the tumor, an MHC type, an Rh parameter, i.e., measuring the antigens of the Rh blood type system, a nutrient parameter, i.e., concentrations of nutrients, a density parameter, i.e., density of the tumor or tumor sample, and a tumor necrosis factor (TNF) parameter, i.e., concentrations or activity of TNF in the tumor or tumor sample, and the presence, concentrations and/or activity of tumor associated protease enzymes.

The step of adjusting the artificial culture medium according to the tumor microenvironment parameters includes, for example, simulating the tumor microenvironment parameters in the artificial culture medium, by adjusting the composition of the artificial culture medium to correspond the tumor microenvironment. The step of treating includes contacting the viable tumor sample or administering to the subject e.g., a checkpoint inhibitor, a cytokine, an antibody or fragment thereof, a cell-based therapeutic agent, radiation therapy, a chemotherapeutic drug, and/or an exosome.

The chemotherapeutic agent is any art-known anticancer drug, including, for example, Bendamustine, Bortezomib, Cabazitaxel, Chlorambucil, Cisplatin, Cyclophosphamide, Dasatinib, Docetaxel, Doxorubicin, Epirubicin, Erlotinib, Etoposide, Everolimus, Gefitinib, Idarubicin, Hydroxyurea, Imatinib, Lapatinib, Melphalan, Mitoxantrone, Nilotinib, Oxiplatin, Paclitaxel, Pazopanib, Pemetrexed, Rapamycin, Romidepsin, Sorafenib, Vemurafenib, Sunitinib, Teniposide, Vinblastine, Vinorelbine, and Vincristine, while suitable antibodies or fragments thereof include Reopro, Kadcyla, Campath, Simulect, Avastin, Benlysta, Adcetris, Cimzia, Rbitux, Prolia, Zevalin, Tysabri, Gazyva, Arzerra, Xolair, Vectibix, Perjeta, Cyramza, Lucentis, Rituxan, Bexar, Yondelis, and/or Herceptin.

The step of quantifying the effect of the treatment includes, for example, generating a validation metric as a function of the effect on the at least one non-tumor cell and/or generating the validation metric as a function of the tumor microenvironment parameters. Optionally, the step of quantifying the effect will also include counting cell numbers of the at least one non-tumor cells.

In an alternate aspect, the step of quantifying the effect includes one or more of, measuring tumor sample mass, measuring tumor sample size, and/or measuring tumor sample volume.

Further, the step of quantifying the effect includes, one or more of, measuring non-tumor cell mass, measuring non-tumor cell size, and/or measuring non-tumor cell volume.

In certain aspects of the invention, the at least one non-tumor cell is a tumor infiltrating lymphocyte, such as at least one of a B-cell, a T-cell, a macrophage, an NK cell and/or an immune competent cell, such as an antigen presenting cell. For example, the at least one non-tumor cell is a healthy cell.

In further aspect of the invention, the inventive method includes placing a portion of the viable tumor sample within an artificial microenvironment chamber. This includes, for example, placing the viable tumor sample into a capsule disposed within the patient. In certain additional aspects of the invention, the artificial microenvironment chamber includes a scaffold suitable as a support for tissue growth.

In another aspect of the invention, the invention also includes a step of optimizing the at least one treatment as a function of quantified non-tumor cell metrics.

In order to better appreciate the present invention, the following terms are defined. Unless otherwise indicated, the terms listed below will be used and are intended to be defined as stated, unless otherwise indicated. Definitions for other terms can occur throughout the specification.

It is intended that all singular terms also encompass the plural, active tense and past tense forms of a term, unless otherwise indicated.

The phrase “consisting essentially of” means that the composition and methods may include additional ingredients and/or steps, but only if the additional ingredients and/or steps do not materially alter the basic and novel characteristics of the claimed composition or method.

A “subject” or “patient” according to the invention is a human subject, such as a human patient in need of anticancer treatment or therapy. In certain aspects, the invention can also be applied in a veterinary practice to any vertebrate animal in need of such treatment. This could include, for example, a non-human primate, a canine, a feline, a porcine or pig, an equine, including a horse, donkey, mule, or zebra, and any other animal for which it is desired to validate an antitumor immune response. When a tumor or other tissue sample or cell sample is obtained from a subject, that subject is the donor subject.

The term “antibody” as used herein is intended to encompass any art known type of natural or engineered antigen-binding protein or polypeptide, which includes at least one antigen specific variable domain (VL, VK or VH, or analogous domains engineered into functional polypeptide binding domains). The antibody can be polyclonal, as elicited by a neoeptitope vaccine, or monoclonal as derived from a hybridoma or engineered as a synthetic monoclonal antibody. Derivatives and fragment of polyclonal and/or monoclonal antibodies are also contemplated to be employed. These include Fc fragments, Fab fragments, single-chain antigen-binding polypeptides, and synthetic polypeptides incorporating two or more binding specificities, and the like.

A “validation metric” as employed herein refers to a qualitative or quantitative confirmation of an anticipated effect or treatment outcome. A qualitative validation metric would be presented in the form of a change in the status of a cell or tissue or subject. For example, identifying that there is a predominant effect of a treatment on a cell type or tissue such as noting that the tumor cells died or that certain immune cells were activated. A quantitative validation metric would be presented in numerical, ratio or percentage format, e.g., some specific percent of all NK cells in the tumor have cytotoxic effect.

The term “viable” as used herein is intended to indicate that the referenced tissue or cell is a living tissue or cell. The phrase, “tumor microenvironment” as used herein is intended to indicate the local environment in which a solid tumor exists, in vivo. For a solid tumor, the tumor microenvironment includes the chemical, ionic, endocrine and physical properties of the tissue, blood and/or plasma. The tumor microenvironment also includes, for example, immune cells, immune factors and signaling molecules such as, e.g., cytokines, and the composition and density of the extracellular matrix that are part of, and that are immediately surrounding, the anatomical site of a solid tumor. The tumor microenvironment also includes the expression and concentrations of tumor associated protease enzymes, both soluble and cell membrane bound. The tumor microenvironment also includes the rate and pressure of local blood perfusion, the gross and microanatomy of the blood vessels and lymphatic drainage that may be present in and around a solid tumor, and pressure and/or shear stresses that may exist within and around the solid tumor.

### DETAILED DESCRIPTION

Accordingly, the invention provides a method of validating an anticancer treatment within an artificial tumor microenvironment comprising:
a) obtaining a viable tumor sample from a subject;
b) acquiring tumor microenvironment parameters from the subject's tumor, i.e., the donor subject, from the viable tumor sample obtained from the donor subject, or from tissue surrounding the anatomical site of the tumor in the donor subject;
c) adjusting an artificial culture medium according to the tumor microenvironment parameters;
d) treating at least a portion of the tumor sample in the adjusted artificial culture medium with at least one treatment; and
e) quantifying an effect of the at least one treatment.

Quantifying step (e) can be measured with respect to at least one non-tumor cell within the portion of the tumor sample, or alternatively, can be measured with respect to the tumor or tumor cell parameters determined at the start of the treating step.

### Obtaining a Tumor or Other Tissue Sample from a Subject

A viable tumor sample, or a sample of tissue adjacent to the tumor, can be obtained from a subject having a tumor or cancer by any art known method. For example, the sample can be surgically removed during exploratory surgery as a biopsy sample. Alternatively, the sample can be obtained during surgical resection of a tumor mass, in whole or in part. In a further alternative, the tumor or other tissue sample is acquired in the form of cells (e.g., CTCs, etc.) recovered from blood or other body fluids, as a plurality of suspended cells or a tissue, and is cultured in vitro or in vivo, to form a 3D tumor mass suitable for the methods of the invention.

A tumor biopsy or excised tumor mass is also contemplated to be obtained with the local blood vessels intact, so that these may be characterized. For example, the endothelial cells of the tumor mass can be characterized with respect to a proposed treatment.

A biopsy or resected sample of tumor tissue is removed and maintained in a culture medium under aseptic conditions. If the artificial 3D tumor tissue is to be grown from dispersed tumor cells, the tumor sample is then dispersed to obtain the included tumor cells by art known methods, e.g., it is washed, minced, and enzyme treated with collagenase and/or hyaluronidase to break up connective tissue. Further washing and centrifugation will separate the cells of the excised tumor tissue from any non-cellular debris.

### Acquiring Tumor Microenvironment Parameters

Broadly, tumor microenvironment parameters can be obtained by analyzing the obtained tumor sample in vitro or in vivo, according to art known methods. For example, Bonutti et al., US20080086072, describe methods for monitoring the various tissue parameters in regions of the body. According to Bonutti et al, in vivo access to a tumor microenvironment is obtained by inserting an endoscope, or other analogous hardware, into the tissue to be measured, while employing art known sensors for detecting required parameters, such as, local pH, moisture, humidity, oxygen level, oxygenase, carbon dioxide level, rate of blood flow or perfusion, nutrient levels, osmolarity, pressure, vascular permeability, blood type, MHC type, Rh type, blood type, nutrient levels, tissue perfusion rates, tissue density, tumor necrosis factor (TNF) levels, tumor associated protease enzyme expression and levels, and the like. Additionally, contemplated tumor microenvironment parameters include determining the presence and/or absence of specific non-tumor cells, and especially immune competent cells. For example, suitable immune competent cells include CD4+ T cells, CD8+ T cells, T helper cells, B cells, NK cells, M1 and/or M2 macrophages, Tregs, MDSC, and dendritic cells.

Simply by way of example, oxygen sensors for measuring oxygen levels (02 partial pressures) in vivo are described by Wilson et al. in U.S. Pat. No. 9,044,179. Methods of measuring tumor tissue perfusion rates are described, for example, by Degani, in US20030211036. Methods of measuring tissue glucose levels in vivo are described, for example by Kollias et al. U.S. Pat. No. 6,505,059

Measurement of the tumor microenvironment is also contemplated to include measuring and characterizing exosomes produced by and/or contained in the tumor tissue, by any art known methods. For example, the exosomes are captured and quantified by an ExoTEST™. The ExoTEST™ analysis is based on a double sandwich ELISA assay designed to detect expression of exosome specific antigenic markers in body fluids or tissue samples. Such markers include, for example, the housekeeping proteins CD63 and Rab-5, and the tumor-associated marker caveolin-1. For example, the ExoTEST™ is conducted by binding to a primary Rab-5 antibody as described by Fais, et al, (U.S. Pat. No. 8,617,806 and U.S. Pat. No. 8,617,806).

Tumor microenvironment determinations are also contemplated to include acquiring omics data from a biopsy or excised tumor sample. For example, the omics data are derived from one or more of the following methods: whole gene sequencing (WGS), whole exome sequencing (WES), transcriptome profiling, including mRNAs, non-coding RNAs and small RNAs (RNAseq), and profiling the proteins expressed by the tumor (proteomics analysis). In addition, the omics data is also contemplated to include metabolomics measurements of the tumor sample.

WGS requires sequencing the entire genomic contents of cell, tissue or organism, including mitochondrial DNA. WGS can be conducted by art known methods, for example, by Illumina dye sequencing (Illumina, Inc.), pyrosequencing, single molecule real time (SMRT) sequencing and/or nanopore sequencing. The data obtained can be in the form of whole genome sequences, or processed by BamBam to record the point by point differences between the tumor and normal cell genomes. According to Sanborn et al., US20120059670, the BamBam method reads from two files at the same time, constantly keeping each BAM file in synchrony with the other and piling up the genomic reads that overlap every common genomic location between the two files. For each pair of pileups, BamBam runs a series of analyses before discarding the pileups and moving to the next common genomic location. By processing these massive BAM files with this method, the computer's RAM usage is minimal and processing speed is limited primarily by the speed that the file system can read the two files.” Thus, a file showing only the differences is produced.

Whole exome sequencing (WES) of tumor samples is contemplated to be provided by art known methods and reagents. These include target-enrichment methods that permit the selective capture of genomic regions of interest from a DNA sample prior to sequencing. Methods include direct genomic selection (DGS) by Basiardes et al., (2005). Nature Methods. 1(2): 63-69. doi:10.1038/nchembio0705-63, Molecular Inversion Probe (MIP), Absalan, et al., 2008, Methods in Molecular Biology 396: 315-330. doi:10.1007/978-1-59745-515-2, and hybrid capture on microarrays, to name several such systems. Commercially available WES systems include, for example, NimbleGen SeqCap Target Enrichment and SeqCap EZ from Roche, Inc. and SureSelect target enrichment from Agilent.

Transcriptome profiling of tumor samples is contemplated to be provided by RNA sequencing and/or quantification, that can be performed by any methods known in the art, and may use various forms of RNA. Some such methods are described, for example, by US 20140148347, and includes ultra-high throughput sequencing approaches, which has high sensitivity and a large dynamic range, as described by Marioni et al. 2008, Genome Res 18(9): 1509-1517; Asmann et al. 2009, BMC Genomics 10: 531. Wang et al. 2009, Nat Rev Genet 10(1): 57-63; and Marguerat et al., 2010, Cell Mol Life Sci 67(4): 569-579.

For example, preferred materials include mRNA and primary transcripts (hnRNA, a/k/a precursor or pre-mRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis. Most typically, RNA quantification and sequencing is performed using qPCR and/or rtPCR based methods, although other methods (e.g., solid phase hybridization-based methods) are also deemed suitable.

Similarly, it is contemplated that proteomics analysis can be performed by all known methods of proteomics analysis. However, particularly preferred proteomics methods include antibody-based methods and mass spectroscopic methods (and especially selected reaction monitoring). Moreover, it should be noted that the proteomics analysis may not only provide qualitative or quantitative information about the protein per se, but may also include protein activity data where the protein has catalytic or other functional activity. Exemplary techniques for conducting proteomic assays are described by U.S. Pat. No. 7,473,532 and U.S. Pat. No. 9,091,651, including immunoassays, mass spectroscopy, Liquid Chromatography-Parallel Reaction Monitoring/Mass Spectrometry (LC-PRM/MS), Liquid Chromatography Selected Reaction Monitoring Mass Spectrometry (LC-SRM-MS), protein array analysis, wherein specific capture reagents of known binding affinity, such as antibodies, are immobilized or spotted onto a support surface in a known positional manner, thus forming a high throughput array for analyzing and detecting proteins in biological materials.

Cancer protease enzymes are markers for cancer diagnosis and are part of the tumor microenvironment that aids in the growth and spread of tumors. These include prostate specific antigen (PSA), matrix metalloproteinases (MMPs), tissue-type plasminogen activator (t-PA), Cathepsin and Type II Transmembrane Serine Proteases.

Cancer related proteases in the tumor microenvironment can be determined by art-known methods, e.g., by determining the levels of RNA that express the proteases in the tumor tissue or individual tumor cells, and/or by using antibody or reagent based enzyme assays for determining in vivo or in vitro enzyme protease activity or expression in the tumor microenvironment.

For example, PSA enzyme activity can be determined by art-known methods, e.g., as described by U.S. Pat. No. 6,764,825 and/or U.S. Pat. No. 5,688,658. There are also commercially available reagents and kits, e.g., human PSA ELISA Kit (KLK3) (ab113327) from abcam (San Francisco, Calif.).

The matrix metalloproteinases (MMP) family includes at least 25 structurally related zinc-dependent proteinases that are secreted into the extracellular space, or anchored on the cell surface, to cleave the extracellular matrix. D. Pei, 2005, Cancer Cell 7(3): 207-8. The MMP alleles can be detected and characterized by RNA expression analysis, e.g., as described for breast cancer by Köhrmann, et al. 2009, (BMC Cancer 9: 188; doi:10.1186/1471-2407-9-188). MMP enzymes are also contemplated to be detected by commercially available reagents. For example, the MMP9 Human ELISA Kit MMP9 Human ELISA Kit Cat. No. KHC3061, and the MMP2 Human ELISA Kit Cat No. KHC3081, both from Thermo Fisher Scientific (Waltham, Mass., USA).

t-PA a serine protease enzyme levels or activity can be detected, for example, by commercially available reagents, e.g., Human Tissue Type Plasminogen Activator ELISA Kit (TPA) (abcam Cat No. ab108914) and tPA Human ELISA Kit Cat No.: BMS258-2 from Thermo Fisher Scientific (Waltham, Mass., USA).

Cathepsin enzyme levels or activity is also contemplated to be detected by commercially available reagents. For example, the InnoZyme™ Cathepsin B Activity Assay Kit, Fluorogenic, Cat. No. CBA001, from Millipore Sigma (Temecula Calif., USA). There are also the Easy ACT™ kits for detecting cathepsin B, cathepsin L and cathepsin K from Leinco Technologies (St. Louis, Mo., USA).

Metabolomics, or metabolic profiles of a tumor sample, is contemplated to be provided by art known methods of detecting and quantifying small molecules as present in tissues, or as extracted from tissues. Such methods can be conducted in vitro, and in certain aspects, in vivo. Generally, analytic methods include, for example, thin layer chromatography (TLC), high pressure liquid chromatography (HPLC), infrared spectroscopy (IR), near-infrared spectroscopy (near-IR), nuclear magnetic resonance (NMR), mass spectroscopy, art known commercially available enzyme specific and immunoassay specific analysis systems, electrodes specific for one or more metabolites, and the like. For example, such methods are described by Kaddurah-Daouk, et al., U.S. Pat. No. 7,005,255, Fernandez, U.S. Pat. No. 8,370,070 and Walker et al., US20080234945.

### Adjusting an Artificial Culture Medium According to the Tumor Microenvironment Parameters

The artificial culture medium is adjusted to conform to the tumor microenvironment parameters obtained according to the above, by adjusting the microenvironment parameters of the artificial tumor to conform to the measured values of the subject's tumor microenvironment. These include, for example, tissue pH, moisture, oxygen level, oxygenase, carbon dioxide level, rate of blood flow or perfusion, nutrient levels, osmolarity, pressure, vascular permeability, blood type, Rh type, cancer associated protease enzymes, levels of nutrients, tissue density, cytokine levels, e.g., TNF levels, exosome numbers and types, detailed omics data, and the like. Additionally, where needed, adjustment of the artificial culture medium may be performed by adding or removing immune competent cells, and especially CD4+ T cells, CD8+ T cells, T helper cells, B cells, NK cells, M1 and/or M2 macrophages, Tregs, MDSC, and dendritic cells, for example, from the subject from which the tumor sample was derived.

The inventive method, wherein the step of adjusting the artificial culturing medium according to the tumor microenvironment parameters comprises adding or removing one or more of the tumor microenvironment elements, as listed above, or by preparing a cell or tissue culture medium to mirror the previously determined tumor microenvironment as found in a subject's tumor.

### Identifying Subject Specific or Tumor Specific Neoepitopes

Once a tumor sample is obtained from a subject, it is also analyzed to identify any neoepitopes that may be present, and that may provide a suitable target or targets for anticancer immunotherapy. In more detail, a cancer neoepitope is, for example, one or more of a cancer-associated or cancer-specific peptide, a nucleic acid, a lipid, a sugar, and/or other type of molecule that the subject reacts to. A cancer neo-epitope is also a combination of a cancer-associated and/or a cancer-specific peptide, a nucleic acid, a lipid and/or a sugar. In certain aspects, a cancer neoepitope is a cancer-associated or cancer-specific peptide, protein and/or nucleic acid identified by a genetic comparison between a cancer cell and a normal cell of a subject. In another aspect, the neoepitope is an HLA-matched neoepitope.

Thus, in one aspect, it is contemplated that prior to providing individualized anticancer treatment, a tumor biopsy is obtained from a subject in need thereof, and omics analysis is performed on the obtained tumor sample. In general, it is contemplated that the omics analysis includes whole genome and/or exome sequencing, RNA sequencing and/or quantification, and/or proteomics analysis by art-known methods. Preferred analytic methods include WGS (whole genome sequencing) and exome sequencing of both tumor and matched normal sample. Likewise, the computational analysis of the sequence data may be performed in numerous ways. In most preferred methods, however, analysis is performed in silico by location-guided synchronous alignment of tumor and normal samples as, for example, disclosed in US 2012/0059670A1 and US 2012/0066001A1 using BAM files and BAM servers. Of course, alternative file formats (e.g., SAM, GAR, FASTA, etc.) are also expressly contemplated herein.

In addition, RNA sequencing and/or quantification can be performed by any methods known in the art, and may use various forms of RNA. For example, preferred materials include mRNA and primary transcripts (hnRNA), and RNA sequence information may be obtained from reverse transcribed polyA⁺-RNA, which is in turn obtained from a tumor sample and a matched normal (healthy) sample of the same subject. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis. Most typically, RNA quantification and sequencing is performed using qPCR and/or rtPCR based methods, although other methods (e.g., solid phase hybridization-based methods) are also deemed suitable. Viewed from another perspective, transcriptomic analysis may be suitable (alone or in combination with genomic analysis) to identify and quantify genes having a cancer and/or subject specific mutation.

Similarly, proteomics analysis can be performed by numerous methods, and all known methods of proteomics analysis are contemplated herein. However, particularly preferred proteomics methods include antibody-based methods and mass spectroscopic methods (and especially selected reaction monitoring). Moreover, it should be noted that the proteomics analysis may not only provide qualitative or quantitative information about the protein per se, but may also include protein activity data where the protein has catalytic or other functional activity. Exemplary techniques for conducting proteomic assays are described, for example, by U.S. Pat. No. 7,473532 and U.S. Pat. No. 9,091,651.

Therefore, tumor-specific neoepitopes are identified against a matched normal sample of a subject, and preferably included in the analysis only if the neoepitope is due to a missense mutation and/or above a minimum expression level (e.g., at least 20%). Additionally, such filtering can be further refined by confirming high transmembraneous expression level of cancer neoepitopes. To facilitate computational analysis, it is contemplated that neoepitopes will be confined to relatively small fragments having a minimum size necessary for antibody binding (e.g., at least 5-6 amino acids) and a maximum size of 20 amino acids (and in some cases longer). Thus, suitable neoepitopes will preferably have a length of between 7-12 amino acids, for example, nine amino acids, including the changed amino acid.

Genomic analysis can be performed by any number of analytic methods. However, especially preferred analytic methods include exome and whole genome sequencing of both tumor and matched normal sample. Likewise, the computational analysis of the sequence data may be performed by numerous methods. However, in particularly preferred methods, analysis is performed in silico by location-guided synchronous alignment of tumor and normal samples as, for example, disclosed in US 2012/0059670A1 and US 2012/0066001A1 using BAM files and BAM servers. It should be noted that any language directed to a computer should be read to include any suitable combination of computing devices, including servers, interfaces, systems, databases, agents, peers, engines, controllers, or other types of computing devices operating individually or collectively. One should appreciate that the computing devices comprise a processor configured to execute software instructions stored on a tangible, non-transitory computer readable storage medium (e.g., hard drive, solid state drive, RAM, flash, ROM, etc.).

The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In certain aspects, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Identification of expression level can be performed by any art-known method. Preferred methods include quantitative RNA (hnRNA or mRNA) analysis and/or quantitative proteomics analysis. Most typically, but not necessarily, the threshold level for inclusion of neoepitopes will be an expression level of at least 20%, and more typically at least 50% as compared to matched normal, thus ensuring that the epitope is at least potentially ‘visible’ to the immune system. Thus, it is generally preferred that the omics analysis also includes an analysis of gene expression (transcriptomic analysis) to so help identify the level of expression for the gene with a mutation. Viewed from another perspective, transcriptomic analysis may be suitable (alone or in combination with genomic analysis) to identify and quantify genes having a cancer and subject specific mutation. There are numerous methods of transcriptomic analysis know in the art, and all of the known methods are deemed suitable for use herein. Taken the above into consideration, it should therefore be appreciated that a subject sample comprising DNA and RNA from tumor and matched normal tissue can be used to identify specific mutations and to quantify such mutations.

Neoepitopes obtained as described above may be subject to further detailed analysis and filtering using predefined structural and expression parameters, and/or sub-cellular location parameters. For example, it should be appreciated that neoepitope sequences are only retained provided they will meet a predefined expression threshold (e.g., at least 20%, 30%, 40%, 50%, or higher expression as compared to normal), or are identified as having a membrane associated location (e.g., are located at the outside of a cell membrane of a cell). Further contemplated analyses will include structural calculations that delineate whether or not a neoepitope is likely to be solvent exposed, presents a structurally stable epitope, etc. Further examples, methods, and neoepitopes are found in International applications WO2016/164833 (filed 8 Apr. 2016) and WO2016/172722 (filed 25 Apr. 2016), both incorporated by reference herein.

Obtaining Neoepitope Peptides and Antibodies

To obtain useful quantities of the neoepitope(s) identified above, it is contemplated that the epitope(s) identified above are prepared in vitro to yield a synthetic epitope, e.g., a synthetic peptide. There are numerous methods known in the art to prepare synthetic peptides, and all known methods are deemed suitable for use herein. For example, peptides with cancer neoepitope sequences can be prepared on a solid phase (e.g., using Merrified synthesis), via liquid phase synthesis, or from smaller peptide fragments. In some other aspects, peptides could also be produced by expression of a recombinant nucleic acid in a suitable host (especially where multiple neoepitopes are on a single peptide chain, optionally with spacers between neoepitopes or cleavage sites).

Therefore, the structure of the synthetic peptides corresponding to the neoepitope sequences may be X-L₁-(Aₙ-L₂)ₘ-Q, in which X is an optional coupling group or moiety that is suitable to covalently or non-covalently attach the synthetic peptide to a solid phase, L₁ is an optional linker that covalently links the synthetic peptide to a solid phase, or to the coupling group. Aₙ is the synthetic peptide having the neoepitope sequence, with A being a natural (proteinogenic) amino acid and n is an integer between 7 and 30, and most typically between 7 and 11 or 15-25. L₂ is an optional linker that may be present, especially where multiple synthetic peptide sequences (identical or different) are in the construct, and m is an integer, typically between 1 and 30, and most typically between 2 and 15. Finally, Q is a terminal group which may be used to couple the end of the synthetic peptide to the solid phase (e.g., to sterically constrain the peptide) or to a reporter group (e.g., fluorescence marker) or other functional moiety (e.g., an affinity marker and/or an immune modulator molecule according to the present invention). Consequently, it should be noted that where the synthetic peptide is used for direct MHC-I binding, the overall length will be between 8 and 10 amino acids. Similarly, where the synthetic peptide is used for direct MHC-II binding, the overall length will be between 14 and 20 amino acids. On the other hand, where the synthetic peptide is processed in the cell (typically via proteasome processing) prior to MHC presentation, the overall length will typically be between 10 and 40 amino acids, with the changed amino at or near a central position in the synthetic peptide. Therefore, and viewed from a different perspective, the synthetic neoepitope may be a short single neoepitope sequence between 7-13 or between 15-25 amino acids, optionally with a linker between the peptide and a solid phase, or a concatemer of more than synthetic neoepitope where a linker is typically interposed between the individual synthetic neoepitopes.

For example, X could be a non-covalent affinity moiety (e.g., biotin) that binds a corresponding binding agent (e.g., avidin) on the solid phase, or a chemical group (with or without spacer) that reacts with the N- or C-terminal amino or carboxyl group of the peptide, or a selectively reactive group (e.g., iodoacetyl or maleimide group) that reacts with a sulfhydryl group in the peptide or linker L₁. L₁ may be used to increase the distance of the synthetic peptide from the solid phase and will therefore typically comprise a flexible linear moiety (e.g., comprising glycol groups, alkoxy groups, glycine, etc.) having a length of equivalent to between about 2-20 carbon-carbon bonds (e.g., between 0.3 nm and 3 nm). Of course, it should also be appreciated that the synthetic peptide may use the solid phase on which the peptide was produced and as such not require a separate coupling group or linker.

Depending on the particular synthetic peptide and coupling method, it should be appreciated that the nature of the solid phase may vary considerably, and all known solid phases for attachment of peptides are deemed suitable for use herein. For example, suitable solid phases include agarose beads, polymer beads (colored or otherwise individually addressable), wall surfaces of a well in a microtiter plate, paper, nitrocellulose, glass, etc. The person of ordinary skill in the art will be readily appraised of a suitable choice of solid phase and attachment chemistry. In further preferred aspects, it is also noted that the solid phase will generally be suitable for protocols associated with phage display methods such as to allow peptides presented on a phage (or other scaffold carrier) to reversibly bind to the solid phase via the synthetic peptide. In still further contemplated uses, it should also be recognized that the solid phase may be a carrier protein used in vaccination (e.g., albumin, KLH, tetanus toxoid, diphtheria toxin, etc.), particularly where the synthetic protein is used as a vaccine in a mammal or as an immunogenic compound in a non-human mammal for antibody production. Likewise, the synthetic protein may also be used as a vaccine or immunogenic compound without any carrier.

To obtain an antibody, or fragment thereof, that binds to the identified neoepitope, the above described synthetic peptide is employed as an antigen, with a suitable adjuvant and/or an expression vector encoding a multi epitope construct, to elicit an immune response in a suitable subject human or other mammalian immunological system.

### Neoepitope Vaccines

In a further aspect of the invention, the immune response to be validated is induced by administering a vaccine that presents the neoepitope to a subject, or to immune cells, in the form of a recombinant expression vector and/or recombinant host cell. A neoepitope is encoded by a nucleic acid expression vector, that when expressed in a suitable host cell, will express and produce a protein or peptide that comprises the neoepitope. Suitable expression vectors include plasmid and/or virus vectors, depending on the selected host cell expression system. For example, an art-known viral vector is an adenovirus vector.

Other suitable protein expression vectors known in the art are selected based upon the expression host (e.g., an expression vector with a mammalian promoter system that is suitable for expression in a human cell line, whereas a yeast or bacterial expression plasmid would be selected if expression in either of these organisms was desired.

In one aspect, the vaccine is formulated with a nucleic acid vector capable of being taken up by host cells of the subject and expressing a neoepitope in vivo the subject in need of such treatment. In another aspect, recombinant host cells are employed to express a neoepitope, or a fusion protein that includes a neoepitope, ex vivo, that is then isolated, purified and formulated as an anticancer vaccine. In a further aspect, such recombinant host cells expressing the neoepitope, or a fusion protein that includes a neoepitope, are directly formulated as an anticancer vaccine and administered as a host cell based vaccine to the subject in need thereof.

Suitable host cells for expressing a vector encoding a neoepitope include bacterial strains, such as Escherichia coli. Suitable yeast host cells include, for example, yeast from the genera of Saccharomyces, Schizosaccharomyces, Kluveromyces, Hansenula, and/or Candida. A suitable species of Pichia includes, for example, Pichia pastoris. A suitable species of Saccharomyces includes, for example, Saccharomyces cerevisiae. Yeast vaccines and methods of preparing yeast vaccine for immunotherapy of cancer are described, for example, by Franzusoff et al. (e.g., U.S. Pat. No. 7,563,447, U.S. Pat. No. 8,343,502, U.S. Pat. No. 8,734,778 and U.S. Pat. No. 9,549,970),

### Treating the Viable Tumor Sample in the Adjusted Culture Medium

The invention is contemplated to include treating the artificial 3D tumor sample with cancer neoepitopes and/or individualized anticancer vaccines, anticancer antibodies, and/or other art known anticancer treatments or modalities, as described above. The step of treating the artificial 3D tumor sample optionally also includes, for example, contacting the tumor sample with a checkpoint inhibitor, a cytokine, a cell-based therapeutic agent, a chemotherapeutic drug, and an exosome. The tumor sample is contemplated to be grown in vitro, or in vivo, e.g., in a capsule embedded in a test animal, or in the donor subject, as part of the inventive method.

In certain aspects of the invention, where the tumor microenvironment is defined by the presence or absence of certain immune competent cells from the patient, and where the capsule retains these cells in the culture environment, the capsule may well be permeable to provide nutrient exchange while retaining the cellular components. Also, the implantation can be at an anatomic site where oxygen supply is limited, thus mimicking hypoxic and/or pH conditions in the microenvironment. Thus, when grown in vivo, the tumor sample is contemplated to be grown and maintained within a capsule that is embedded or implanted in a test animal, or in the donor subject.

Preferably, the implanted in vivo capsule comprises a protective device or material that serves to isolate the tumor sample from the cellular and tissue environment of the host animal, while allowing nutrients and waste products to pass. Optionally, while the tumor sample is maintained within the implanted capsule, supplementary chemical or biological factors are injected or infused into the capsule, e.g., via implanted microtubing, to adjust the microenvironment of the tumor sample to correspond to the microenvironment of the native tumor.

The implanted in vivo capsule may comprise or contain a scaffold formed by a matrix of interconnected growth surfaces spaced at regular intervals according to e.g., U.S. Pat. No. 9,220,731, as described in more detail below as part of an in vitro culture device.

The implanted in vivo capsule is contemplated to be composed, in whole or in part, of a membrane or other suitable barrier, that allows the infusion of nutrients and biological substances into the capsule, and waste products to diffuse out, but prevents the movement of living cells in or out, avoiding, in most aspects, the requirement for additional hardware for pumping or infusing a culture medium through the capsule and/or through any scaffold enclosed by the capsule.

For in vitro cultivation, the tumor sample is grown and maintained in an appropriate culture medium, including a culture system that functions as a “GMP in-a-box system,” using the Austrianova's Cleanroom setup, or using a device as described by US20150065359, or using the culture systems taught by US20160108358, published on Apr. 21, 2016 and US20160083682, published on Mar. 24, 2016, or using a continuous culturing device as described by U.S. Pat. No. 9,220,731.

For example, as described by US20160108358, a device for culturing 3 dimensional (3D) tissue samples in vitro is a culture device comprising (a) a scaffold formed by a matrix of interconnected growth surfaces spaced at regular intervals, in order to define open spaces along X, Y and Z Cartesian axes; and (b) fluid distribution means at opposite ends of the scaffold providing: an inlet for fresh culture fluid, and an exit for used culture fluid containing waste, from the growth areas. The spacing of the interconnected growth surfaces is arranged to optimize uniform directional flow through and around the growth surfaces.

The fluid distribution means at the inlet and the exit of the growth areas permits an adequate flow to each growth surfaces. The fluid distribution is analyzed using computational fluid dynamics and key metabolite utilization analysis to assure that the cells are not subject to detrimental growth conditions.

In one aspect, the fluid distribution means distributes the incoming flow of fresh nutrients and gasses to the growth surfaces. The cross-sectional area of the distribution device channels and the number of channels can be adjusted to facilitate the uniform distribution to the growth surfaces, depending on the shape of the growth surfaces and the total number of cells supported by the growth surfaces.

In another aspect, the culture device includes a matrix of interconnected growth surfaces, defined by the interconnection of multiple fibers or three-dimensional structures, in an organized and repetitive manner, which can incorporate any number of facets or surface artefacts utilized to encourage or enhance the attachment and growth of cells.

The three-dimensional structures forming the matrix can be cylindrical, rectangular, hexagonal, or any other shape or combination of shapes and the surfaces may be smooth or textured. In a practical aspect of the invention, the scaffold is formed by a matrix of interconnected growth surfaces spaced at regular intervals around a central support. The open spaces formed by the interconnection of the structures, are equal or larger than 0.7 mm and smaller than 3 mm, or equal or larger than 0.9 mm and smaller than 3 mm. The spacing in this aspect is greater than 1.0 mm, but can be altered as required by the need for physical strength of the scaffold. In a still further aspect of the present invention, the interconnected growth surfaces are spaced at regular intervals equal or larger than 1.0 mm and less than 2.0 mm.

Spacing is a characterizing feature of the 3D culture device. The variability of the parameter around the above range allows to optimize the flow of medium throughout the scaffold and, at the same time, to impart an adequate solidity to the 3D structure for all the devices independently from their final shape and dimension. The open spaces formed by the interconnection of the growth surfaces create the organized characterizing structure of the 3D culture device which differs from the porous structure of the device known from the prior art. The shape of the scaffold is preferably cubic, but it could be another shape, for example, cylindrical or anatomically correct.

Preferably, the 3D culture device includes a large number of interconnected growth surfaces uniformly arranged to create large open areas that limit the maximum number of cells per cubic volume facilitating the easy vascularization of the growth areas.

The 3D culture device can be made of any biocompatible material. Biocompatible materials are any biocompatible organic polymer or mixture thereof as well as blends or mixtures of biocompatible organic polymers with biocompatible organic or inorganic non-polymeric compounds. Non limitating specific examples of components of the biocompatible material useful in the 3D culture device are polycaprolacton, polyethylene oxide—terephthalate, polyamide, poly-L-lactic acid, polyglycolic acid, collagen, fibronectin, hydroxyapatite, etc.

In a practical aspect, the 3D culture device further comprises an aseptically sealed housing that can be disassembled at the completion of the culture period. The aseptic housing can include a sealed removable cover, an inlet distribution means, an optional exit distribution means, and the necessary support means required to locate and secure the growth surfaces in the 3D culture device. The housing can be in the form of a rectangle, cylinder or any other shape necessary to hold the culture device and provide additional features for aseptic removal of the scaffold.

An inlet fluid distribution device is connected to an input side of the scaffold and having a common fluid inlet that is fluidly coupled with a plurality of inlet distribution conduits through which input fluid is delivered to and distributed through the open spaces of the scaffold.

An outlet fluid collection device is connected to an output side of the scaffold and having a plurality of outlet conduits that are fluidly coupled with a common fluid outlet that collects output fluid from the open spaces of the scaffold.

A housing is coupled to the inlet fluid distribution device and the outlet fluid collection device, and that houses the scaffold, inlet fluid distribution device, and outlet fluid collection device. In this arrangement, the housing, scaffold, inlet fluid distribution device, and outlet fluid collection device comprise a single work piece.

The 3D culture device of can be produced in a single step process. Alternatively, a 2D layer can be produced first, and then the single 2D layers can be assembled one over the others to form the 3D culture device according to the present invention. The final dimension of the 3D continuous culture device will depend on the number of assembled 2D layers.

The 3D culture device can be efficiently used for culturing tumor cells and/or tumor tissue samples into a 3D tissue. Once the cells have grown into a 3D tissue, the media flow may be stopped and the tissue can be used or preserved for future use. The 3D culture device can be efficiently used also for culturing cells directly into the body. In fact, the device can be implanted into a test animal or subject so that the culturing is effected in vivo.

By using the 3D culture device tumor tissue may be grown in a controlled environment on a biodegradable scaffold. The large open areas formed by the interconnection of the growth surfaces allows them to be exposed to a uniform flow of medium and to prevent fouling during the growth process. In particular, fouling or blockade of the growth surface by gas bubbles during the growth process is prevented.

Moreover, with the 3D culture device, culture conditions are monitored continuously and any departures from the desired conditions are automatically corrected and alarmed. This provides conditions necessary to maintain cells in their undifferentiated state, to minimize the maximum cell density and the associated toxic necrosis, and to provide an environment that is not diffusion limited for key nutrients and gasses.

Systems and equipment for the 3D culture of tumor tissue samples and analysis of the resulting 3D tissues are also commercially available. For example, PerkinElmer (Waltham, Mass., USA) sells a wide range of hardware and tools for the 3D culture of tissues, including PerkinElmer CellCarrier Spheroid Ultra Low Attachment Microplates, Insphero GravityTRAP™ ULA Plate, GravityPLUS™ Plate, Insphero GravityPLUS™ Hanging-Drop Spheroid Culture System, with analysis provided by e.g., the PerkinElmer Opera Phenix High-Content Screening System. The AlgiMatrix® 3D Cell Culture System are plates and an algae matrix for 3D cell culture that is available from Thermo Fisher Scientific (Waltham, Mass., USA). 3D cultures can also be formed by growing tumor cells in a medium, incorporating the expanded cells into a bioink (a gel or gel-like support matrix) and printing a 3D model tumor ex vivo. 3D-bioprinting systems are commercially available from a number of sources, including, e.g., the Bioplotter Manufacturer Series and Developer Series from EnvisionTEC (Detroit, Mich., USA), and the BioBot1 from BioBots, Inc. (Philadelphia, Pa., USA).

The inventive method, further comprising placing the portion of the tumor sample within an artificial microenvironment chamber, such as a capsule inserted within the subject. In certain aspects, the artificial culturing microenvironment also contains a scaffold material or matrix that supports the 3D growth of the cultured tumor cells.

Quantifying an Effect of the Treatment on a Non-Tumor Cell Within the Tumor

One or more non-tumor cells can be monitored for cellular changes in response to treatment Non-tumor cells that are contemplated to be monitored include, for example, an immune competent cell, a tumor infiltrating lymphocyte and/or an antigen presenting cell. Such a tumor infiltrating lymphocyte can be, for instance, a B-cell, a T-cell, a macrophage, an NK cell, an M1 macrophage, an M2 macrophage, a dendritic cell, and a myeloid-derived suppressor cell (MDSC). The T-cell can be, for example, a suppressor T-cell, a Th1 cell, a Th2 cell, a Th17 cell, a regulatory T-cell, or an anergic T-cell. The non-tumor cell can be present in a mass of tissue that includes other non-tumor cells.

The effects of the anticancer treatment on such non-tumor cells found within the tested viable tumor sample are contemplated to be quantified by any art-known methods. Broadly, the level of immune cell infiltration by tumor infiltrating lymphocytes (TIL) in biopsy samples, for example, and cell counts, can be obtained by light microscopy of tissue sections stained with a standard hematoxylin and eosin (H&E) stain and quantifying the immune cells present in the tissue sample by general histological appearance. More particular methods include, for example, staining sections of the tumor sample with tagged antibodies that bind to known immune cell antigenic markers (e.g., CD68, CD4, CD11, etc.). Changes in immune cell antigenic marker counts or intensities in samples of 1 the tumor is monitored for example, for changes in the presence of the antigenic markers, as a function of time of each particular treatment, against a baseline control of pretreatment sample or untreated tumor sample.

These methods can be automated, as described, for example, by Galon et al., US20150153349, by employing apparatus for automated slide staining, digital image capture and digital image processing to quantify the presence and type of immune cells in multiple tissue samples.

Alternatively, the presence of non-tumor cells in the tumor sample are contemplated to be quantified and monitored before and after application of a test treatment by employing comparative genomic analysis. For example, as described by Robins, et al., U.S. Pat. No. 9,181,590, DNA is extracted from a portion of the tumor sample before, during and/or after the tumor is treated. The presence of markers for non-tumor cells, e.g., T-cells, is then monitored for changes in the presence of the markers specific for the type of non-tumor cell being monitored, as a function of time of each particular treatment, against a baseline control of pretreatment or untreated tumor.

The above described methods for quantifying non-tumor cells are contemplated to include measuring sample mass, measuring sample size, and measuring sample volume. The above methods for quantifying non-tumor cells are also contemplated to optionally include generating a validation metric as a function of the treatment that is applied and/or as a function of the tumor microenvironment parameters. The non-tumor cells can also be monitored for (clonal) growth/propagation, cytotoxic or cytolytic effects on tumor cells, production of antibodies or suitable T cell receptors, secretion of cytokines and/or chemokines, or other growth factors, tumor suppressor factors, as will be appreciated by the artisan.

What is claimed is:

1. A method of validating an anticancer treatment within an artificial tumor microenvironment comprising:
(a) obtaining a viable tumor sample from a tumor present in a subject;
(b) acquiring tumor microenvironment parameters from the subject's tumor, i.e., the donor subject, from the viable tumor sample obtained from the donor subject, or from tissue surrounding the anatomical site of the tumor in the donor subject;
(c) adjusting an artificial culture medium according to the tumor microenvironment parameters;
(d) treating at least a portion of the tumor sample in the adjusted artificial culture medium with at least one anticancer treatment; and
(e) quantifying an effect of the at least one anticancer treatment.

2. The method of claim 1, wherein the viable tumor sample comprises a plurality of suspended cells or a tumor tissue sample.

3. The method of claim 1, wherein the step of obtaining the viable tumor sample comprises obtaining a sample of vasculature at or adjacent to the in vivo anatomical location of the tumor.

4. The method of claim 1, wherein the step of acquiring tumor microenvironment parameters comprises taking a metabolomics measurement of the viable tumor sample.

5. The method of claim 1, wherein the step of acquiring tumor microenvironment parameters comprises acquiring omics data of the viable tumor sample.

6. The method of claim 5, wherein the omics data are derived from at least one of whole gene sequencing (WGS), whole exome sequences (WES), transcriptome profiling including mRNAs, non-coding RNAs and small RNAs (RNAseq), and profiling the proteins expressed by the tumor (proteomics analysis).

7. (canceled)

8. The method of claim 1, wherein the step of acquiring tumor microenvironment parameters comprises taking an in vivo measurement of the tumor microenvironment.

9. The method of claim 1, wherein the step of acquiring tumor microenvironment parameters comprises taking a measurement of the properties of the vasculature of the tumor.

10. The method of claim 1, wherein the step of acquiring tumor microenvironment parameters comprises taking a measurement of the exosomes present in the tumor.

11. The method of claim 1, wherein the tumor microenvironment parameters are measured in the tumor tissue and comprise one or more of an oxygen parameter, a pH parameter, an Rh parameter, a blood type, a nutrient parameter, a density parameter, a TNF concentration, and the presence, concentrations and/or activity of tumor associated protease enzymes.

12. The method of claim 1, wherein the step of adjusting the artificial culturing medium according to the tumor microenvironment parameters comprises simulating the tumor microenvironment parameters in the artificial culturing medium.

13. The method of claim 1, wherein the step of treating that is to be validated is selected from the group consisting of, administering to the subject, or contacting the viable tumor sample, with one or more of a checkpoint inhibitor, a cytokine, an antibody or fragment thereof, a cell-based therapeutic agent, a chemotherapeutic drug, radiation therapy, and/or an exosome that exhibits anticancer properties.

14-16. (canceled)

17. The method of claim 1, wherein quantifying step (e) is measured with respect to at least one non-tumor cell within the portion of the tumor sample, or is measured with respect to the tumor or tumor cell parameters determined at the start of the treating step.

18. The method of claim 17, wherein the step of quantifying an effect of the at least one anticancer treatment includes:
(a) generating a validation metric as a function of the effect,
(b) counting cell numbers of the at least one non-tumor cell,
(c) at least one of measuring sample mass, measuring sample size, and measuring sample volume, and
(d) at least one of measuring non-tumor cell mass, measuring non-tumor cell size, and measuring non-tumor cell volume.

19. The method of claim 18, further comprising generating the validation metric as a function of the tumor microenvironment parameters.

20. The method of claim 17, wherein the at least one non-tumor cell comprises an immune competent cell.

21-27. (canceled)

28. A method of validating an anticancer treatment within an artificial tumor microenvironment comprising:
(a) obtaining a viable tumor sample from a tumor present in a subject;
(b) acquiring tumor microenvironment parameters from the subject's tumor, i.e., the donor subject, from the viable tumor sample obtained from the donor subject, or from tissue surrounding the anatomical site of the tumor in the donor subject;
(c) adjusting an artificial culture medium according to the tumor microenvironment parameters;
(d) treating at least a portion of the tumor sample in the adjusted artificial culture medium with at least one anticancer treatment; and
(e) quantifying an effect of the at least one anticancer treatment.

29. The method of claim 28, wherein the viable tumor sample comprises a plurality of suspended cells or a tumor tissue sample.

30-34. (canceled)

35. The method of claim 28, wherein the step of acquiring tumor microenvironment parameters comprises taking an in vivo measurement of the tumor microenvironment.

36-44. (canceled)

45. The method of claim 28 any of the preceding claims, wherein the step of quantifying an effect of the at least one anticancer treatment includes:
(a) generating a validation metric as a function of the effect,
(b) counting cell numbers of the at least one non-tumor cell,
(c) at least one of measuring sample mass, measuring sample size, and measuring sample volume, and
(d) at least one of measuring non-tumor cell mass, measuring non-tumor cell size, and measuring non-tumor cell volume.

46-54. (canceled)
